(19) United States
(12) Patent Application Publication
(10) Pub. No.: US 2021/0308289 A1
(43) Pub. Date: Oct. 7, 2021

(54) INACTIVATION OF HIGHLY RESISTANT INFECTIOUS MICROBES AND PROTEINS WITH UNBUFFERED HYPOHALOUS ACID COMPOSITIONS

(71) Applicant: BRIOTECH, INC., WOODINVILLE, WA (US)

(72) Inventors: Daniel James TERRY, WOODINVILLE, WA (US); Jeffrey Francis WILLIAMS, WOODINVILLE, WA (US)

(73) Assignee: BRIOTECH ,INC., Woodinville, WA (US)

(21) Appl. No.: 16/313,054

(22) PCT Filed: Jun. 22, 2017
(86) PCT No.: PCT/US2017/038838

§ 371 Date: Jun. 29, 2020

Related U.S. Application Data
(60) Provisional application No. 62/353,483, filed on Jun. 22, 2016.

(51) Int. Cl. A61L 2/00; A01N 59/00; A61L 2/18; A61L 2/22

(52) U.S. Cl. CPC A61L 2/0088; A61L 2/22; A61L 2/18; A01N 59/00

(57) ABSTRACT

Methods for true sterilization of an object, methods for inactivating an infectious protein, and methods for inactivating a microbial pathogen using a bufferless, electrolyzed, hypohalous acid composition.

### CROSS-REFERENCE TO RELATED APPLICATION

This application is a continuation of International Patent Application No. PCT/US2017/038838 with a filing date of Jun. 22, 2017, designating the United States, now pending, and further claims priority to U.S. provisional application 62/353,483 with a filing date of Jun. 22, 2016. The content of the aforementioned applications, including any intervening amendments thereto, are incorporated herein by reference.

### BACKGROUND OF THE INVENTION

The emergence of multi-drug resistant disease microbes in the last several decades has ushered in a new era of infectious disease challenges. There is an urgent need for improved means of preventing and controlling exposure of people and animals to invasive pathogenic microbes, particularly those that can survive for long periods in the environment, or that resist conventional decontamination procedures. The latter have proven inadequate both for effective, high-level disinfection of the most durable infectious agents on surfaces, instruments or devices, and in regard to their safety for operators, patients, and the environment. Current measures fail without the incorporation of long and impractical exposure times, elevated temperatures or pressures, or hazardous or corrosive solutions or vapors. Although all known infectious agents of disease do eventually succumb to physical and chemical extremes, such measures are inconvenient or even hazardous for practical applications, and can damage valuable equipment. They do not provide ready solutions to growing health risks from well-recognized microbial pathogens, or from self-replicating proteins, increasingly associated with degenerative neurological diseases in humans, domestic and wild animals. As a result of these flaws tragic, fatal iatrogenic transmission incidents have come about from ineffective decontamination measures applied to instruments and devices used on unsuspecting patients.

Despite the advances made in the inactivation of disease agents, a need exists for convenient, cost-effective, entirely non-hazardous methods applicable to high level decontamination/inactivation of disease agents that pose challenges for infection control measures today. The present invention seeks to fulfill this need and provides further related advantages.

### SUMMARY OF THE INVENTION

Methods and compositions are disclosed for inactivating infectious agents to a high degree, after short exposure periods, and under conditions that are mild and harmless to surfaces, instruments, equipment and operating personnel. These methods and compositions are strikingly different in character and duration from those conventionally applied to the decontamination of items and surfaces that are suspected of containing or having been exposed to highly resistant agents. In the past, suitable levels of confidence in the complete inactivation of all infectious agents required harsh and prolonged high temperature treatments (for example, using pressurized steam at 132° C. for 30 minutes) after prior immersion in caustic and corrosive chemical agents such as 2N sodium hydroxide or concentrated sodium hypochlorite solutions (10,000-40,000 mg/L) for periods of 1-2 hours. These procedures create significant hazards to personnel handling large volumes of dangerous chemical solutions, and exposing costly autoclaving equipment to vapors created by extensive heat treatment of the decontamination target. By contrast, the compositions disclosed herein allow inactivation of resistant agents at room temperature (20° C.), in short contact periods (seconds to an hour) without necessity for additional, high temperature post-chemical exposure treatment. The compositions disclosed herein do not involve expensive, corrosive or impractical compositions or procedures. Prior methods, while proven to degrade the infectivity of all known agents, do not readily find a place in the real-world practice of high level decontamination in healthcare or other arenas, such as carcass preparation and food processing, or countermeasures against bioterrorism, where concerns about the entire spectrum of infectious agents are appropriate.

The inactivating constituents are preferentially stable, aqueous solutions of pure hypohalous acid (hypochlorous acid, or hypobromous acid) in which the contaminated article or tissue or bodily fluid is suspended for periods up to one hour at 20° C. or higher in order to achieve reductions in infectivity of 6 Log Reduction Value (LRV) or greater. The hypohalous acid concentrations required for maximal inactivation are optimally in the 150-300 mgs/L range. Lesser concentrated solutions, or exposures for shorter periods, can nonetheless result in significant reductions in the infectivity of target agents. At these optimal concentrations the inactivating solutions are not corrosive or toxic to mammalian cells in vitro, or to human or animal skin or mucous membranes, including nasal, oral and conjunctival epithelia. These specifications for effective degradation of the infectious potential of highly resistant microbial agents, such as bacterial and fungal spores, and non-enveloped, capsid-protein coated viruses, and infectious proteins, are compatible with practical demands of healthcare and environmental disinfection and decontamination. They permit adoption of the disclosed methods for widespread use in combatting transmission of all resistant disease agents. They are compatible with commercial viability of the methods for everyday use, without concerns for the integrity and utility of treated surfaces, devices, and equipment, or for the health and safety of personnel responsible for executing the methods on a routine basis.

The invention provides the advantage that high level decontamination can be accomplished in one step for spores, viruses and multi-drug resistant vegetative forms of microbial disease agents and infectious proteins, unlike certain previous approaches that required addition of conventional disinfecting or denaturing formulations or procedures after the primary exposure to decontamination measures.

The use of stable, pure hypohalous acid solutions allows for highly convenient methods of exposure of contaminated surfaces, equipment, devices, clothing or personnel to inactivating fogs or mists of these solutions into confined spaces. This procedure ensures dispersion of the active compositions into crevices and microenvironments, even onto personnel who are suspected of having been contaminated by infectious tissues or bodily fluids, without concerns for the toxicity or corrosiveness which accompany prior methods. It also obviates concerns about reliable efficacy of the means of decontamination that are always associated with unstable hypochlorous acid preparations.

While the preferred inactivation procedure makes use of aqueous solutions of hypohalous acids at concentrations in the 150-300 mgs/L range, the active constituents are compatible with formulations as gels or viscous fluids. These may be applied to target surfaces to ensure prolonged and intimate contact with the necessary levels of active halogen species.

The overall aspect of the preferred solutions used for pathogen inactivation disclosed herein is the exposure of targeted surfaces, equipment, devices, tissues or bodily fluids to solutions of hypochlorous acid within the range of pH 3.2-6.0, and preferentially pH 3.8-5.0 with an optimal range of pH 4.0-4.3, having an Oxidation Reduction Potential (ORP) of +1000, and preferentially +1100 and optimally +1138 millivolts (my), containing from 0 up to about 2.0% by weight chloride salt, preferentially from about 0.85% to about 2.0% by weight chloride salt (e.g., NaCl) for periods up to one hour. The solution of HOBr is preferentially within the range of pH 3-8, with an optimum of about pH 7, with an ORP of +900, preferentially +1000 my, and containing from 0 to about 2.0% by weight chloride salt, preferentially from about 0.85% to about 2.0% by weight chloride salt (e.g., NaCl). The HOCl solutions are sufficiently stable to ensure that optimal specifications can be maintained at these levels, or at levels sufficient to provide for high efficacy in the inactivation of infectious agents, for a period of three to five or more years when stored in sealed vessels. HOBr is preferentially made at time of use from such a stable solution of HOCl, but may be used for four to six weeks after its de novo formation following the addition of an equivalent of one equivalent of NaBr or KBr to an equivalent (HOCl) of the stable HOCl solution.

A further advantage of the invention is the suitability of the inactivation solutions for treatment of potentially contaminated tissues that may be useful in transplantation procedures such as corneal grafting, dura grafts, or other tissues or organs that may be required for restoration of functions in a recipient host, or may be used for cosmetic manipulation of the recipient (e.g., bovine collagen injections or implants).

A further advantage of the invention is the suitability of the inactivation solutions for the pre-treatment of implanted devices, electrodes, sensors, and the like into the human body for the purposes of restoring or assisting in preservation of functions in the recipient host.

A further advantage of the invention is the suitability of the inactivation solutions for neutralization of infectious agents that may be used as instruments of bioterrorism, and of certain chemical agents that may be used in the conduct of chemical warfare.

A further advantage of the invention is the potency of the stable, pure hypohalous acids in disrupting adherent mixed populations of microbes that are resistant to conventional antimicrobial agents including high concentrations of hypochlorite bleach.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a Raman spectrum of a representative hypochlorous acid formulation (BrioHOCL™) useful in the methods of the invention.

FIG. 2 compares oxidative chlorine concentrations in ppm in aliquoted samples of a representative HOCl formulation useful in the methods of the invention (BrioHOCL™) stored at either room temperature (RT) or 70° C.

FIGS. 3A and 3B compare serial measurements of pH (3A) and ORP (3B) in aliquoted samples of a representative HOCl formulation useful in the methods of the invention (BrioHOCL™) stored at either room temperature (RT) or 70° C.

FIG. 4 compares serial measurements of Cl ppm (Log n) in aliquoted samples (52) of a representative HOCl formulation useful in the methods of the invention (BrioHOCL™) stored at 52° C.

FIG. 5 compares serial measurements of Cl ppm (Log n) in aliquoted samples (70) of a representative HOCl formulation useful in the methods of the invention (BrioHOCL™) stored at 70° C.

FIG. 6 is the UV/Vis absorption spectrum of a representative HOBr solution useful in the methods of the invention adjusted to pH 9 with sodium hydroxide.

FIG. 7 is the Raman spectrum of a representative HOBr solution useful in the methods of the invention illustrating the characteristic waveform peak at 615 cm⁻¹.

FIG. 8 illustrates titrable bromine (Br) (ppm) versus time of representative HOBr solutions useful in the methods of the invention after storage at room temperature in glass containers.

### DETAILED DESCRIPTION OF THE INVENTION

The present invention relates to methods and compositions for the inactivation of highly resistant infectious agents, on surfaces or in suspension, in biological fluids or tissues, upon exposure to solutions, gels, mists or vapors containing stable, unbuffered solutions of hypohalous acids.

Methods of Use for Hypohalous Acid Compositions

Methods for using hypohalous acid compositions are provided.

In one aspect, the invention provides a method for true sterilization of an object, comprising contacting an object to be sterilized with a bufferless, electrolyzed, hypohalous acid composition.

As used herein, “true sterilization” refers to the inactivation of all forms of microbial life, including microbial disease agents of bacterial, viral, fungal or protozoal origin, alone or in combination, as well as non-living infectious disease agents known as prion proteins, which resist conventional sterilizing measures. Conventional sterilization is understood to be the inactivation of all forms of microbial life including microbial disease agents of bacterial, viral, fungal or protozoal origin, but is not understood to include the inactivation of infectious proteins. Because the methods and compositions of the invention are effective in inactivation of microbial life and non-living infectious disease agents (e.g., prion proteins), the methods and compositions are effective for true sterilization.

As used herein, “disinfection” denotes a lesser level of antimicrobial inactivation than sterilization, and specifically directed to the reduction in numbers of disease agents responsible for infections in humans, animals, and plants, but not encompassing forms of life that do not participate in infectious disease processes.

As used herein, the term “bufferless, electrolyzed, hypohalous acid composition” refers to a composition of a hypohalous acid that is not buffered (does not include a pH buffer) that is electrolytically-generated. As used herein, the terms “bufferless” and “unbuffered” are used interchangeably.

Bufferless, electrolyzed, hypohalous acid compositions useful in the methods of the invention include solutions that are commercially available from Briotech Inc., Woodinville Wash. under the designation BrioHOCL™ and BrioHOBR™, which are bufferless, electrolyzed, solutions of hypochlorous acid (HOCl) and hypobromous acid (HOBr), respectively.

Commercially available BrioHOCL™ and BrioHOBR™ are representative bufferless HOCl and HOBr solutions, respectively, useful in the methods of the invention.

In certain embodiments, the ionic strength of these representative bufferless HOCl and HOBr solutions (BrioHOCL™ and BrioHOBR™, respectively) is increased to provide novel HOCl and HOBr solutions that are effective for enhancing the inactivation of prions. Bufferless HOCl and HOBr solutions of increased ionic strength (e.g., about 2% by weight chloride salt based on the total weight of the composition) are useful for elevating the level of inactivation of the proteins to a higher degree for a given time and dose of exposure. Given that prion diseases are uniformly 100% fatal after onset, the highest level possible of inactivation is desirable for a given dose and time of exposure of prion contaminated items or tissues.

In certain embodiments, the object is a surface. Suitable surfaces include medical instruments, surgical instruments, laboratory surfaces, implanted devices. Other surfaces that can be sterilized by the method of the invention include environmental surfaces in confined spaces such as hospital rooms, laboratories, clinics, operating theaters, dental offices, post-mortem rooms, mortuaries, animal necropsy facilities, abattoirs, animal housing quarters, bedding, meat processing facilities, surgical or diagnostic instruments, devices, and tools used in these environments, and inanimate devices used as implants for therapeutic or diagnostic purposes, and whole carcasses or corpses of animals or patients or parts thereof, processed in any such environments.

In other embodiments, the object is a biological sample. Suitable biological samples include bodily fluids and tissues. Representative biological samples include intact tissues of animal or human origin, or derivations of tissues used for diagnostic purposes, or therapeutically or cosmetically as grafts or implants (e.g., skin, cornea, dura mater, collagen), or the biological fluids conventionally associated with these tissues or their derivations, such as blood, saliva, sputum, cerebrospinal fluids, nasal secretions, ocular fluids, or urine or excreta that make contact with the sampled or prepared tissues or their associated organs.

In another aspect, the invention provides a method for inactivating an infectious agent, comprising contacting an infectious agent with a bufferless, electrolyzed, hypohalous acid composition.

As used herein, the term “inactivating” or “inactivation” refers to the elimination to a practically and statistically important extent (e.g., substantial elimination) of the infective capacity of an infectious microbe or other infectious agent. The term “inactivated” refers to an infectious microbe or other infectious agent that has had its infective capacity substantially eliminated.

As used herein, the term “infectious agent” refers to infectious microbial agents and infectious agents that are not associated with microbes (e.g., non-living infectious agents, such as prions).

As noted above, infectious microbial agents may be of bacterial, viral, fungal or protozoal origin, acting alone or in combination.

Infectious agents that are not associated with a microbial structure recognizable as a form of life include infectious proteins that are devoid of genetic information in the form of DNA or RNA, but capable of self-replication. Exemplary infectious proteins include prions. Representative prions effectively inactivated by the methods and compositions of the invention include the prion agents of Creutzfeldt Jakob Disease, Bovine Spongiform Encephalopathy, Chronic Wasting Disease, Scrapie, and human neurodegenerative diseases, such as Alzheimer's Disease, Parkinson's Disease, and Amyotrophic Lateral Sclerosis, among others.

Representative infectious agents that are effectively inactivated by the compositions and methods of the invention include viruses, bacteria, fungi, and protozoa. In addition to these microbes, infectious agents that are effectively inactivated by the compositions and methods of the invention include infectious proteins, such as self-replicating proteins.

In one embodiment, the infectious agent is an infectious microbe. Representative microbes include viruses, bacteria, fungi, or protozoa.

In another embodiment, the infectious agent is an infectious protein.

Representative infectious proteins include self-replicating proteins.

In a further embodiment, the infectious agent is an airborne particulate. In certain of these embodiments, the airborne particulate is inactivated in the air by, for example, a spray, mist, fog, or aerosol that includes the bufferless, electrolyzed, hypohalous acid composition.

In a further aspect, the invention provides a method for inactivating an infectious protein, comprising contacting an infectious protein with a bufferless, electrolyzed, hypohalous acid composition.

In one embodiment, infectious protein is an infectious self-replicating protein.

In one embodiment, the infectious protein is a prion. In certain embodiments, the prion is an agent of Creutzfeldt Jakob Disease, Bovine Spongiform Encephalopathy, Chronic Wasting Disease, Scrapie, Alzheimer's Disease, Parkinson's Disease, and Amyotrophic Lateral Sclerosis.

In a further aspect, the invention provides a method for inactivating a microbial pathogen, comprising contacting a microbial pathogen with a bufferless, electrolyzed, hypohalous acid composition.

As used herein, the term “microbial pathogen” refers to pathogens that are microbes, including bacteria of Gram negative types (e.g., Acinetobacter baumannii, Escherichia coli, Escherichia coli 0157 Pseudomonas aeruginosa, Salmonella choleraesuis, Shigella flexneri, Escherichia coli NDM-1, Klebsiella pneumonia, Yersinia enterocolitica, Proteus vulgaris, Listeria), bacteria of Gram positive types (e.g., Bacillus subtilis, Staph epidermidis, MRSA (Staph. aureus), Enterobacter cloacae, Enterococcus VRE), fungi (e.g., Candida albicans, Aspergillus niger) and viruses (e.g., Coronavirus [Human, OC43]).

In one embodiment, the microbial pathogen is a Gram negative bacteria. In another embodiment, the microbial pathogen is a Gram positive bacteria. In a further embodiment, the microbial pathogen is a fungi. In certain embodiments, the microbial pathogen is a virus.

In certain of the above methods, the composition is a solution, a spray or fog or mist or aerosol of droplets (e.g., micronized droplets in the submicron size range and aerosolized droplets), a gel, or a viscous liquid.

In certain of the above methods, contacting with the composition comprises contacting from one second to several hours (e.g., one to six hours).

In certain of the above methods, contacting with the composition comprises contacting at room temperature.

In certain of the above methods, contacting with the composition comprises contacting at a temperature in the range from about room temperature to about 80° C.

In certain of the above methods, the hypohalous acid composition is a hypochlorous acid composition.

In certain of these embodiments, the hypohalous acid composition is an aqueous hypochlorous acid composition having a hypochlorous acid concentration from about 5 to about 500 mg/L, a pH from about 3.2 to about 6.0, an oxidative reduction potential (ORP) of about +1000 millivolts, and containing from about 0.85% to about 2.0% by weight chloride salt based on the total weight of the composition.

With regard to oxidative reduction potential (ORP), in certain embodiments, the specified value defines an ORP range; for example, “about +1000 millivolts” defines a range of +/−50 millivolts.

In other of these embodiments, the hypohalous acid composition is an aqueous hypochlorous acid composition having a hypochlorous acid concentration from about 80 to about 300 mg/L, a pH from about 3.8 to about 5.0, an oxidative reduction potential (ORP) of about +1100 millivolts, and containing from about 0.85% to about 2.0% by weight chloride salt based on the total weight of the composition.

In further of these embodiments, the hypohalous acid composition is an aqueous hypochlorous acid composition having a hypochlorous acid concentration from about 80 to about 300 mg/L, a pH from about 4.0 to about 4.3, an oxidative reduction potential (ORP) of about +1138 millivolts, and containing from about 0.85% to about 2.0% by weight chloride salt based on the total weight of the composition.

In other of the above methods, the hypohalous acid composition is a hypobromous acid composition.

In certain of these embodiments, the hypohalous acid composition is an aqueous hypobromous acid composition having a hypobromous acid concentration from about 10 to about 300 mg/L, a pH from about 3 to about 8.5, an oxidative reduction potential (ORP) of about +1000 millivolts, and containing from about 0.85% to about 2.0% by weight chloride salt based on the total weight of the composition.

In other of these embodiments, the hypohalous acid composition is an aqueous hypobromous acid composition having a hypobromous acid concentration from about 5 to about 350 mg/L, a pH of about 7 to about 8, an oxidative reduction potential (ORP) of about +900 millivolts, and containing from about 0.85% to about 2.0% by weight chloride salt based on the total weight of the composition.

In certain embodiments, the chloride salt is an aqueous soluble chloride salt selected from sodium chloride, potassium chloride, magnesium chloride, and ammonium chloride. In certain embodiments, the chloride salt is sodium chloride.

In certain embodiments, the composition contains about 2.0% by weight chloride salt based on the total weight of the composition. In certain embodiments, the composition contains about 2.0% by weight sodium chloride based on the total weight of the composition.

The composition does not contain a detectable amount of aqueous oxidative chlorine other than HOCl. As used herein, “oxidative chlorine” refers to all oxidizing chlorine species (e.g., HOCl, molecular chlorine, chlorate, chlorite, hypochlorite) detectable by, for example, repetitive-scan Raman spectroscopy. In certain embodiments, the composition includes <200 ppm aqueous oxidative chlorine. In other embodiments, the composition includes <100 ppm aqueous oxidative chlorine. In further embodiments, the composition includes <50 ppm aqueous oxidative chlorine. It will be appreciated that for HOBr solutions, the composition does not contain a detectable amount of aqueous oxidative bromine other than HOBr detectable by, for example, repetitive-scan Raman spectroscopy (e.g., <200 ppm aqueous oxidative bromine, <100 ppm aqueous oxidative bromine, <50 ppm aqueous oxidative bromine).

In certain embodiments, the hypohalous acid is hypochlorous acid and the composition has a shelf life of useful inactivation efficiency up to about 5 years in a sealed container. In other embodiments, the hypohalous acid is hypobromous acid and the composition has a shelf life of useful inactivation efficiency of from about four to about six weeks in a sealed container. As used herein, the term “shelf life” refers to the composition's retention of sufficient oxidative hypohalous acid concentration and/or ORP to provide for reliable inactivation of infectious agents to the degree useful in the required application.

The hypohalous acid composition does not include a hypohalous acid stabilizer. The hypohalous acid composition does not include a mono- or di-phosphate sodium or potassium buffer, a carbonate buffer, periodate, divalent metal cation, organic heterocyclic compound, hydrochloric acid, hydrobromic acid, or a chemical entity conventionally used as a halogen stabilizer to enhance the stability of a hypohalous acid solution in storage.

Hypohalous Acid Compositions

In a further aspect, the invention provides bufferless, electrolyzed, hypohalous acid compositions.

In certain embodiments, the bufferless, electrolyzed, hypohalous acid composition, comprises a hypohalous acid and a chloride salt in an amount from about 0- to about 2.0% by weight based on the total weight of the composition. In certain of these embodiments, the chloride salt is an amount from about 0.85 to about 2.0% by weight based on the total weight of the composition.

In certain embodiments, the hypohalous acid is hypochlorous acid.

In certain of these embodiments, the composition comprises hypochlorous acid at a concentration from about 5 to about 500 mg/L, and has a pH from about 3.2 to about 6.0, and an oxidative reduction potential (ORP) of about +1000 millivolts.

In other of these embodiments, the composition comprises hypochlorous acid at a concentration from about 80 to about 300 mg/L, and has a pH from about 3.8 to about 5.0, and an oxidative reduction potential (ORP) of about +1100 millivolts.

In further of these embodiments, the composition comprises hypochlorous acid at a concentration from about 80 to about 300 mg/L, and has a pH from about 4.0 to about 4.3, and an oxidative reduction potential (ORP) of about +1138 millivolts.

In other embodiments, the hypohalous acid is hypobromous acid.

In certain of these embodiments, the composition comprises hypobromous acid at a concentration from about 10 to about 300 mg/L, and has a pH from about 3 to about 8, and an oxidative reduction potential (ORP) of about +1000 millivolts.

In other of these embodiments, the composition comprises hypobromous acid at a concentration from about 5 to about 350 mg/L, and has a pH of about 7, and an oxidative reduction potential (ORP) of about +900 millivolts.

In certain embodiments, the chloride salt is an aqueous soluble chloride salt selected from sodium chloride, potassium chloride, magnesium chloride, and ammonium chloride. In certain embodiments, the chloride salt is sodium chloride.

The HOCl compositions do not contain a detectable amount of aqueous oxidative chlorine other than HOCl. The HOBr compositions do not contain a detectable amount of aqueous oxidative bromine other than HOBr.

In certain embodiments, the hypohalous acid is hypochlorous acid and the composition has a shelf life of useful inactivation efficiency up to about 5 years in a sealed container.

In other embodiments, the hypohalous acid is hypobromous acid and the composition has a shelf life of useful inactivation efficiency of from about four to about six weeks in a sealed container.

The hypohalous acid composition does not include a hypohalous acid stabilizer. The hypohalous acid composition does not include a mono- or di-phosphate sodium or potassium buffer, a carbonate buffer, periodate, divalent metal cation, organic heterocyclic compound, hydrochloric acid, hydrobromic acid, or a chemical entity conventionally used as a halogen stabilizer to enhance the stability of a hypohalous acid solution in storage.

The composition may be formulated to suit the desired application. In certain embodiments, the composition is formulated as a solution, a spray or fog or mist or aerosol of droplets (e.g., micronized droplets in the submicron size range and aerosolized droplets), a gel, or a viscous liquid.

The following is a description of representative hypohalous acid compositions of the invention and their utility.

In general, formulations containing hypochlorous acid (HOCl) along with other aqueous forms of chlorine, are known to be effective antimicrobial agents with proven antiviral, antibacterial, antifungal, and antiprotozoal properties that are useful in disinfection measures applied in human and animal health, and horticulture and Examples 1 and 3 below). Although HOCl is unstable and impure when produced under conventional conditions, crude mixtures containing HOCl may be generated on-site for short-term applications in all these fields of use (USDA Directive 710. 21, 2017). The useful life of these conventional electrolytically prepared solutions is frequently measured in hours. Stabilizing additives can extend the useful life of these preparations to days or weeks depending on the nature of the adjunctive components of the formulations and the methods used for their storage.

Exacting manufacturing processes dependent upon the careful adjustment of the pH of pure solutions of sodium hypochlorite can furnish HOCl with stability that permits prolonged storage, even for periods up to two years. This stability enhances its utility for certain medical applications, but the careful process controls required make the product costly. This restricts its use to medical procedures that can support the pharmaceutical expense levels involved. Manufacture of HOCl by electrolysis has heretofore been unable to generate aqueous formulations with sufficient stability for a wider array of practical uses without the incorporation of buffering systems, and/or a range of stabilizing entities, including metal cations, periodate, phosphate buffers, carbonate buffers, and organic compounds with halogen stabilizing abilities. These solutions may be enhanced in their utility by special packaging for improved storage. Prior to these adjustments to electrolytically-generated HOCl solutions, there were no successful stabilized formulations of this active component in pure solution uncontaminated by either non-hypohalous acid constituents, or other aqueous species of halogens.

All of the additives and chemical stabilizers conventionally employed to support the maintenance of HOCl in active form over practically useful storage periods depend on the presence of other species of aqueous chlorine, such as hypochlorite and chlorite/chlorate, or chlorine, depending on the chemical intervention chosen, or lead to their appearance in the solution as a result of the onset of decay. Many of these constituents contribute toxic effects on cells and tissues to the formulations that limit their usefulness in medical procedures. Aqueous species of halogens other than the hypohalous acids, HOCl and HOBr, all deliver detrimental and often corrosive impacts on environmental surfaces that make them less than ideal for practical purposes. Furthermore, by adjusting the conditions surrounding HOCl in particular, as the most commonly desired hypohalous acid, in order to enhance its shelf life on storage, the potency of the HOCl component is undermined. The resulting antimicrobial efficacy of electrolytically-generated HOCl products becomes therefore a blend of contributions from HOCl, hypochlorite, chlorate, chlorine dioxide, and other aqueous Cl species, if the product pH is being adjusted upwards into the neutral range or higher. Some products are purported to contain additional non-chlorine based activity attributed to other oxidants such as ozone, peroxides, or to short-lived free radicals in solution. When electrolytic products are adjusted into the low pH range (about 3 or below) using mineral acids or carbonic acid the main source of antimicrobial efficacy is aqueous molecular chlorine. This condition is associated with serious hazards arising from off-gassing of molecular chlorine gas, a dangerous respiratory poison for humans and all animals. Recent patents and applications stress the instability of HOCl, and propose adjustments to the process controls and electrolytic cell designs intended to enhance stability, with final product compositions including significant active contributions from constituents other than HOCl. There are corresponding deleterious impacts on the potency of these electrolytically-generated solutions of HOCl, leading to less than optimal efficacies compared to the known potency of the uncontaminated hypohalous acid.

Hypochlorous acid (HOCl) is the conjugate acid of hypochlorite (OCL), and is produced naturally in pure form in vivo by neutrophils in mammals, and in the heterophils of birds to inactivate pathogens within phagocytic vesicles. HOCl in solution is a weak acid (pKa about 7.5). This contrasts with the high alkalinity of household hypochlorite bleach (˜pH 12). Preparations of HOCl uncontaminated by other aqueous halogen species are therefore compatible with applications for which bleach is damaging and hazardous to users, and to the surfaces to which it is applied. Stable, pure HOCl formulations in the form of BrioHOCL™ can be applied directly to the skin and mucous membranes, including conjunctival, oral and genital mucosae, and used as cosmetics, and as topical therapeutics for humans and domestic animals. Hypobromous acid (HOBr) is the conjugate acid of hypobromite, and is produced naturally in eosinophils of mammals via enzymatic pathways similar to those used to generate HOCl. In this case, intracellular bromide ion is oxidized to HOBr rather than chloride ion in the case of HOCl. HOBr has a pKa higher than HOCl. This permits its availability in solution at pH levels higher than those suitable for HOCl, and there are conditions where this characteristic may allow for superior suitability of HOBr over HOCl (e.g., in modifying gelling agents that are unstable at pHs below 7.5-8.0).

HOCl molecules in water are neutral, but aqueous solutions maintain a high positive Oxidation-Reduction Potential (ORP), demonstrable by insertion of millivoltmeter electrodes that will register my potentials typically in the 1100+ range for BrioHOCL™, for example. The measurement of ORP has become accepted as an indicator of the disinfecting capability of active chlorine solutions. The extreme reactivity of the chlorine atom in HOCl leads to known and rapid interactions with a wide range of chemical groups, including oxidation and chlorination reactions with amino acids, lipids and sulfur-containing structures. Many different possibilities arise as to the mechanisms of antimicrobial activity expressed by HOCl solutions, but specific means whereby the infectivity of any particular pathogen is destroyed remain unknown. Nonetheless, there is ample evidence of multiple sites of vulnerability to HOCl in a wide range of proteins and other cellular constituents described in the primary biochemistry literature. This makes it reasonable that HOCl should interact with those specific sites when they are expressed in proteinaceous components of infectious agents of concern in contemporary healthcare, such as in the capsids of resistant small non-enveloped viruses, or as components of infectious proteins themselves.

HOCl and HOBr are known to express a potency in chemical and anti-infective agent reactions that rises to two or more orders of magnitude higher than that of the corresponding hypochlorite and hypobromite entities found in aqueous solutions at pH levels in the alkaline range. Hypochlorite and hypobromite solutions are used for decontamination against a wide range of pathogens, including bacterial and fungal spores, non-enveloped virus particles (some of which are amongst the most difficult microbes to inactivate), protozoan cysts, and even prions that function as infectious proteins. Thus prolonged incubation of prion-contaminated items in concentrated sodium hypochlorite bleach is accepted as a disinfecting measure for this purpose. Likewise, hypobromite solutions have been shown to have inactivation efficacy against prion proteins responsible for bovine transmissible spongiform encephalopathy (BSE, also known as Mad Cow Disease). However, extended exposure of inanimate objects to corrosive solutions of hypochlorite or hypobromite causes damage that may make the practice entirely unacceptable or cause it to be applied only as a last resort, absent alternatives. Similarly, the corrosive effects of these solutions are hazardous to users, and contribute to the unwillingness to use these effectors of inactivation routinely in healthcare institutions and other settings.

At the other end of the scale, acidified electrolyzed solutions of sodium chloride contain aqueous chlorine species that have been shown to have rapid and high level inactivation capacities for a wide range of infectious particles, including bacterial and fungal spores; there is demonstrable activity against infectious prion proteins of Creutzfeldt Jacob Disease (CJD). However, at the extremes of pH (2.6) used for these prion decontamination procedures, there is a predominance of aqueous elemental chlorine as the major oxidant, along with hydrochloric acid (HCl) and some hypochlorous acid. It has been determined that most of the oxidant efficacy under these conditions is attributable to elemental chlorine. The anti-prion efficacy of these formulations is therefore also by inference a function of aqueous chlorine itself. There are hazards associated with the production and handling of this product, including to personnel, in addition to the presence in the formulation of hydrochloric acid.

The efficacy of extreme alkaline or acidic solutions versus prions has attracted interest because of their emerging significance as causes of an increasing number of neurodegenerative disorders in animals and man. Prion diseases, or transmissible spongiform encephalopathies (TSEs), are fatal, untreatable, and transmissible neurodegenerative diseases of many mammalian species. In humans, prion diseases include sporadic, variant and genetic forms of Creutzfeldt-Jakob disease (sCJD, vCJD and gCJD) as well as a number of other disorders. Prion diseases of other species include classical bovine spongiform encephalopathy (cBSE), scrapie in sheep, goats and rodents, and chronic wasting disease of cervids. All mammalian prion diseases share an underlying molecular pathology that involves the conversion of the hosts' normal form of prion protein, (e.g., PrP^{C}), to a misfolded, aggregated, infectious and pathological form (e.g., PrP^{Sc}).

There is recent recognition that pathological forms of proteins that become altered in their conformation are associated with a wider spectrum of diseases than those classically recognized as resulting from transmissible prions, such as CJD, BSE, Scrapie and CWD. Thus now included in the list of diseases that may result from conformationally-altered or misfolded proteins are Alzheimer's Disease, Parkinson's Disease, Frontotemporal Dementia and other neurodegenerative disorders, along with Diabetes Type II, Multiple Systemic Atrophy, and other conditions in which identifiable, abnormally folded proteins may be causative.

All these prions are unusual, compared to other types of pathogens, in that they lack a pathogen-specific nucleic acid genome, and are particularly resistant to biochemical, chemical, physical (e.g., heat, U/V light) or radiological inactivation. As a result, prions resist complete inactivation under conditions that are typically used in healthcare, the food industry, and agriculture to inactivate other types of disease agents, such as glutaraldehyde, peracetic acid, and gaseous agents such as chlorine dioxide or vaporized hydrogen peroxide. Indeed, current recommendations are that extremely harsh chemical treatments such as 1-2 N sodium hydroxide, 20-40% household bleach (about 12,000-24,000 mg/L sodium hypochlorite), prolonged (up to 60 min) autoclaving at an unconventionally high temperature of 132° C. and/or prolonged exposure to incinerator temperatures be used to decontaminate biological materials or solid surfaces that may be contaminated with prions. An anti-prion reagent that was developed and registered with the USEPA as a commercial disinfectant (Environ™ LpH™, an acidic phenolic disinfectant) proved impractical for wide use, and was removed from the US market. In general, it has been determined that all such treatments may not only be hazardous to the user, but can also be incompatible with, or not applicable to, instruments, equipment or surfaces that may require prion decontamination. There is an urgent need for effective high-level decontamination methods that are more safely and broadly applicable to the entire spectrum of resistant infectious agents, including transmissible proteins. The availability of effective, practical inactivation methods for routine use on potentially contaminated tools, instruments, tissues and environmental surfaces would seriously reduce the risks of iatrogenic disease transmission.

Concentrated corrosive solutions, such as lye, or concentrated household bleach act only slowly to degrade the infectivity of resistant agents that take the form of proteins. Moreover, many traditionally used sterilants—defined as agents that inactivate all known forms of microbial life, not only those associated with infections, such as peracetic acid and stabilized hydrogen peroxide and plasmas, are ineffective at prion inactivation, even after prolonged exposure times. It has therefore been generally accepted that conformationally abnormal, misfolded prions are intrinsically resistant to aggressive chemical attack from virtually all directions.

The methods and compositions disclosed herein offer a significant and unprededented departure from that established position. The stable unbuffered HOCl formulation of the invention exhibits rapid potent efficacy against suspensions of a wide range of microbial organisms and infectious agents that are resistant to conventional disinfectants, or susceptible only after prolonged contact times. Its conversion to HOBr at the time of use permits further enhancement of the potency of the hypohalous acid solution versus highly resistant disease agents (see Examples 3 and 4).

HOCl and HOBr covalently modify a number of different amino acid side chain moieties on proteins that are exposed to hypohalous acids, including thiols, amines and aromatic amino acids, all of which are known to be present in infectious prion proteins. Hypohalous acids are most highly reactive to sulfur (S)-containing amino acids, and S-containing amino acids are present in prion proteins, including a single intramolecular disulfide bond between amino acid chains in classical ‘scrapie’ prions. Lysine and other amino acid residues in proteins are particularly susceptible to oxidation to generate chloramines and bromamines. For example, tyrosine side chains can be chlorinated by HOCl, forming 3-Cl-Tyr and 3,5-Cl-Tyr. Dimerization of tyrosine to di-Tyr results from HOCl exposure because phenoxy radicals are generated. Dimerization leads to protein cross-linking within and between molecules harboring the phenoxy radical. These changes are capable of altering the conformation of proteins, and rendering them incapable of expressing intrinsic biological functions. These range from enzymatic activity, to ligand-binding affinity, to template-seeding activity in the case of infectious proteins, without necessarily denaturing the proteins or affecting their solubility, or fragmenting the amino acid backbones. Certain changes resulting from exposure of infectious prions to agents that affect their conformation and seeding capabilities are influenced by the molar concentrations of inorganic salts in the environment.

The present invention provides convenient, cost-effective, entirely non-hazardous methods and compositions applicable to high level decontamination/inactivation of disease agents that pose challenges for infection control measures today. Use of the compositions does not result in damage to surfaces, devices, equipment, and does not require heat, elevated pressure, or prolonged exposures to, or immersion in, toxic or corrosive solutions or vapors. The preferred aqueous solutions of pure hypohalous acids disclosed herein are sufficiently safe and non-toxic to allow for application at full strength to human skin and mucous membranes with no adverse effects whatsoever.

In certain embodiments, the compositions described herein “comprise” the specified components. It is understood that compositions that comprise the specified components may further include other unspecified components. In other embodiments, the compositions “consist essentially of” the specified components and do not include unspecified components that materially alter the characteristics of the composition. In further embodiments, the compositions “consist of” the specified components and do not include any unspecified components.

While the present invention has been described with reference to the demonstrable utility of proprietary unbuffered electrolytically-prepared solutions of HOCl and HOBr, it should be understood by those skilled in the art that certain equivalents may be substituted without departing from the spirit and scope of the invented methods. Modifications may be made to adapt to particular disinfecting and sterilizing decontamination circumstances in accomplishing the objectives, spirit and scope of use of the invented methods. All such modifications are intended to fall within the scope of the invention herein described. The invention constitutes the use of unbuffered, stable, hypochlorous acid or hypobromous acid solutions, uncontaminated with either extraneous additives or other species of aqueous halogens, for the purpose of rendering non-infectious all highly resistant forms of microbial life and other infectious agents by means of exposure of the agents to aqueous solutions, gels, or vapors of micronized droplets of these solutions that are entirely innocuous upon exposure to human skin or mucous membranes.

As used herein, the term “about” refers to +/−10% of the numerical value specified for the parameter.

The following examples are provided for the purpose of illustrating, not limiting the invention.

### EXAMPLES

### Materials and Methods

BrioHOCL™ was supplied by Briotech Inc., Woodinville, Wash. Briefly, HOCl results from brute force electrolysis of an aqueous solution of sodium chloride so as to provide at the anode conditions that attract and stabilize reaction products that form HOCl. The end-product is a solution with a range of pH on packaging and storage of 3.8-4.5 at warehouse environmental temperatures (3.5° C. to 35° C.), an ORP of +1100 my, a salt (NaCl) concentration of either 0.85% or 1.8-2% by weight, and a free chlorine concentration of 250-300 mg/L at the time of production. No adjustments are ever made to this HOCl solution by the addition of buffers, metal ions, organic heterocyclic halogen stabilizers or pH modifiers of any sort, at any level. Details of conditions of storage for purity and stability studies are included in the pertinent Examples below.

Hypobromous acid (HOBr) was prepared by the exposure of one equivalent of aqueous bromide ion (as NaBr) to one equivalent of unbuffered electrolytically-generated HOCl. This solution was prepared fresh for use in tests for inactivation of highly resistant microbial organisms.

Active Chlorine Measurement

Hach reagent kits for Total Chlorine (Hach Company, Loveland, Colo.) were used for determination of the active chlorine (Cl) content of the BrioHOCL™ formulation, after validation by comparison of manual iodometric and digital titration results on 33 samples (six replicates each). Thereafter the digital Hach device was used (4 replicates per sample) to measure active Cl in all samples used for antimicrobial efficacy testing.

Titrable chlorine (Cl) concentrations were also measured in archived commercially prepared product samples at Briotech, Woodinville, Wash., (oldest 34 months), and to establish the titratable Cl trends in a serially sampled lot of BrioHOCL™, stored in sealed about 100 mL aliquots in HDPE bottles at 21° C., and prepared specifically for this purpose. All other BrioHOCL™ samples used throughout these studies were derived from routine production electrolysis runs at the manufacturing plant. Product from each lot was stored in different vessel types (100 ml up to 4 L bottles, and 220 L barrels, all HDPE) in uncontrolled temperature warehouse environments (3.5° C. to 35° C.). Small vessels were sealed with aluminum caps, and drum lids were tightly sealed to avoid exposure to air (known to be deleterious), but no optimization of storage conditions was attempted for materials used herein.

The pH, Oxidation Reduction Potential (ORP in my) and conductivity were recorded for all samples using a Hach Multi Parameter meter (Model HQ40d). ORP targeted at production was +1140 my, at pH 3.9. Starting active Cl concentrations were varied in production lots during electrolysis, depending on intended applications. Generally, these values ranged between 175 and 350 ppm active Cl, with background NaCl concentrations of either 0.85% or 1.8 up to 2% by weight, according to intended use.

UV/Vis Spectrophotometry

Test solutions were loaded into 1 mL quartz cuvettes, and spectra obtained using a BioMate 3S UV-Visible Spectrophotometer. The instrument was blanked using Nanopure water, and test solutions consisted of undiluted BrioHOCL™ at selected time points in the sequential sampling of product stored at room temperature. Absorbance was measured from 190 to 400 nm, with peak absorbance for HOCl registered at 238 nm in the ultraviolet range. Test solutions of HOBr showed an absorbance peak in the ultraviolet range at 260 nm, with no detectable presence of HOCl 5 minutes after the addition of NaBr.

Raman Spectroscopy

Spectra were obtained using a Renishaw InVia Raman microscope. Spectra were observed using an excitation wavelength of 785 nm with undiluted BrioHOCL™ in a 1 mL quartz cuvette. The acquisition time for each scan was 20 seconds, and 100 acquisitions were accumulated. A deionized water blank was scanned in the same manner, and subtracted from the test sample data using Igor software. The same procedure was followed in examining the spectroscopic characteristics of HOBr solutions which were prepared fresh for this purpose.

High Level Disinfection and Biofilm Disruption Evaluation

Details of the methods employed for the evaluation of the high level disinfecting properties, and biofilm disruption properties of Briotech hypochlorous acid solutions are included in the pertinent Example sections below.

### Examples 1-5

### Characterization of Representative Hypohalous Formulations

The following examples are put forth to provide those of skill in the art with a complete description of the characterization of the hypohalous acid solutions with respect to their most important novel and useful attributes. These include the absence of contaminating aqueous halogen species or extraneous stabilizing entities upon production and after storage, their stability under a variety of storage conditions and temperatures, their efficacy in the inactivation of resistant infectious agents, and their safety upon human exposure. The examples are not intended to limit the scope of what the inventors regard as the invention, nor do they represent all the experiments that have been done to demonstrate the utility of the methods disclosed herein.

### Example 1

### Purity of Representative HOCl Solutions (BrioHOCL™) and Effects of Storage

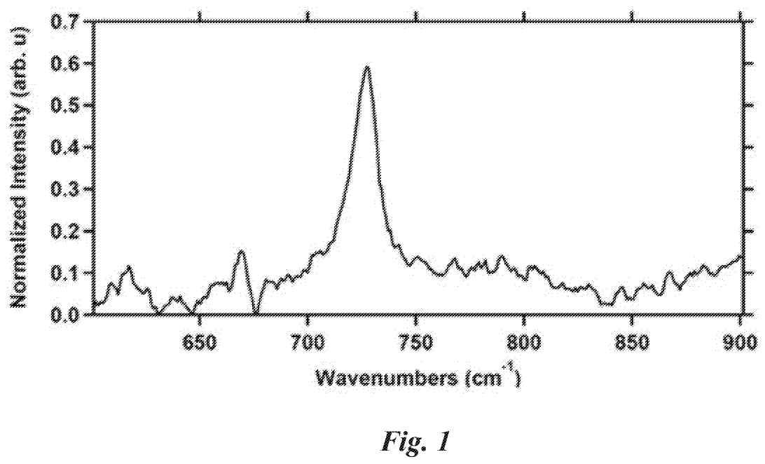

Over a period of more than two years samples of freshly prepared, unbuffered electrolytically generated BrioHOCL™ were collected as aliquots of about 100 mL, and examined by Raman Spectroscopy. These samples consistently revealed a shift peak to wavenumber 728/cm (FIG. 1) corresponding to HOCl only (Nakagawara S, Goto T, Nara M, Ozawa Y, Hotta K, Arata Y (1998). Spectroscopic characterization and the pH dependence of bactericidal activity of the aqueous chlorine solution. Analytical Sciences, 14(4):691-8). In a sample stored for 14 months at room temperature the same profile was revealed by Raman Spectroscopy.

These results indicate that the preparations contained only HOCl. There was no indication of peaks attributable to other chlorine species such as Cl₂, ClO₂, OCl⁻, or OCl₃. Other aqueous chlorine species would have become evident under the conditions of the spectroscopy as peaks >0.3 intensity units between 640 and 870. Spectrophotometric analysis of the representative HOCl formulation (prepared by Briotech) revealed no evidence of the presence of hypochlorite or chlorate in either fresh preparations or those sampled after prolonged storage. These solutions contained no additives such as buffering or stabilizing entities of any nature.

### Example 2

### Stability of Representative HOCl Solutions and Effects of Storage

The purpose of the first experiment was to determine the measurable changes in samples of HOCl exposed to a high temperature that would be expected to degrade conventional preparations. Six samples from lots of BrioHOCL™ (unbuffered) prepared 3-9 months previously and warehouse-stored at uncontrolled temperatures were exposed to an incubator temperature of about 80° C. for 24 hours. The ORP my potentials of the samples were respectively, before and after heating: Sample 1, 1029 my and 1020 my; Sample 2, 1044 my and 1030 my; Sample 3 1060 my and 1040 my; Sample 4, 1057 my and 1030 my; Sample 5, 1040 my and 1040 my; and Sample 6, 1030 my and 1020 my. There was an average decline of only 18.5% in the free chlorine contents of these heated samples. The results indicated that the electrolytically-generated unbuffered HOCl had an unexpected tolerance of high temperatures that would be expected to lead to rapid degradation of conventional hypohalous acid solutions.

### Example 3

### Stability of Representative HOCl Solutions and Effects of Storage

Additional aliquots of BrioHOCL™ (about 100 mL each) were then prepared and sealed for storage in glass or HDPE containers at room temperature, 52° C., or 70° C. The latter were immersed in water baths in which the temperature of the water was adjusted accordingly. Aliquots removed for analysis were discarded once tested, and were not returned to the storage conditions for further study. Raman Spectroscopy, iodometric Cl titrations, UV-visible spectrophotometry, and ORP measurements were used to characterize serially these samples of electrolytically-generated pure unbuffered HOCl (pH 4) made from NaCl and water only. There were no detectable changes in oxidative Cl levels (ppm), ORP (+mv), or pH in HOCl solutions maintained in glass containers at 52° C. for 38 days. After 28 days at 70° C. in glass containers oxidative Cl ppm declined from 190 ppm to 151 ppm, but ORP remained constant, while pH rose to 4.3. In comparison, in HDPE at 52° C., the active chlorine decreased by 53 ppm over 38 days and the pH rose to 5.3, though the ORP remained constant. No oxidative aqueous Cl species other than HOCl were detected in any stored samples by spectroscopy or spectrophotometric analysis.

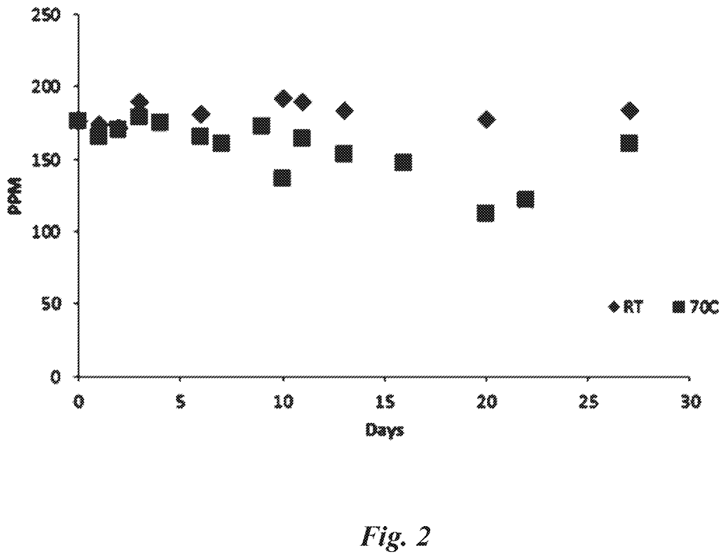

FIG. 2 compares oxidative chlorine concentrations in ppm in aliquoted samples of a representative HOCl formulation useful in the methods of the invention (BrioHOCL™) stored at either room temperature (RT) or 70° C.

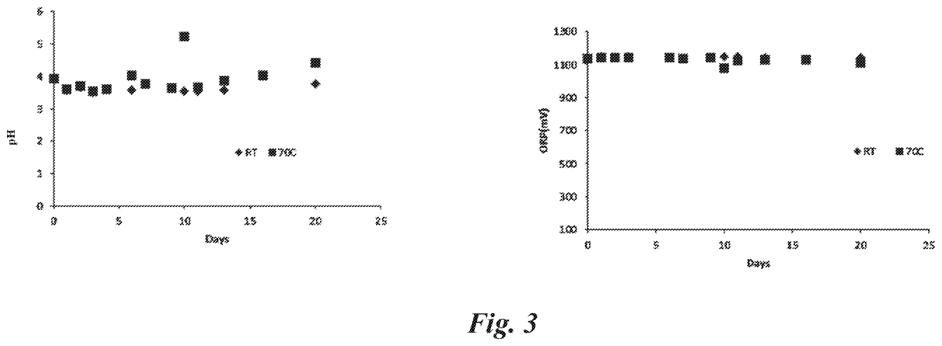

FIGS. 3A and 3B compare serial measurements of pH (3A) and ORP (3B) in aliquoted samples of a representative HOCl formulation useful in the methods of the invention (BrioHOCL™) stored at either room temperature (RT) or 70° C.

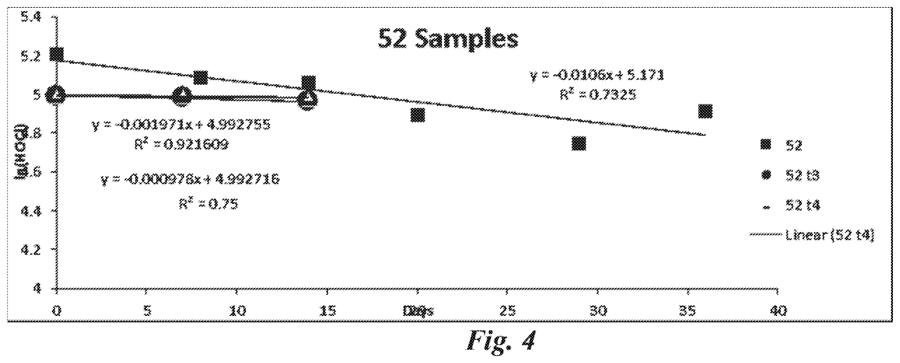

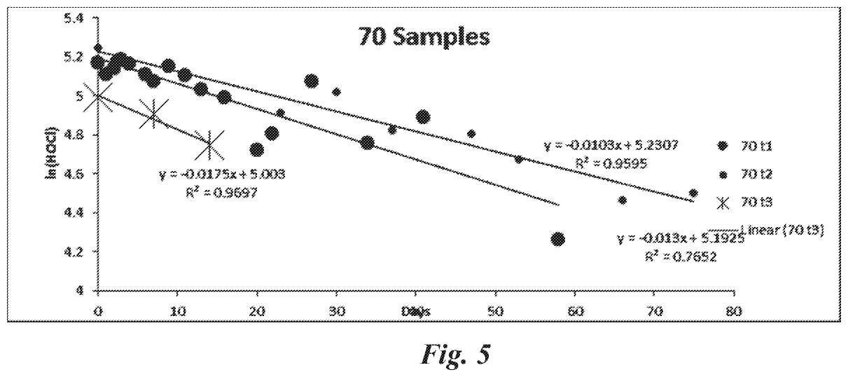

Data from the analysis of replications of the high temperature storage conditions used for determination of stability shown in FIGS. 4 and 5 permit the calculation of a half-life at 52° C. of 460 days, and at 70° C. of 51 days. These correspond to an equivalent half-life at RT of in excess of 5 years in each case (Nicoletti et al. (2009). Brazilian Dental Journal, 20, No. 1).

FIG. 4 compares serial measurements of Cl ppm (Log n) in aliquoted samples (52) of a representative HOCl formulation useful in the methods of the invention (BrioHOCL™) stored at 52° C.

FIG. 5 compares serial measurements of Cl ppm (Log n) in aliquoted samples (70) of a representative HOCl formulation useful in the methods of the invention (BrioHOCL™) stored at 70° C.

Stability in practice enables reliable utility of the solutions in their ability to retain and express sufficient oxidative halogen, and a sufficiently high ORP to deliver the expected antimicrobial efficacy in use against infectious agent contaminants in the environment or on other targeted sites of application (such as instruments, tissue samples or grafts).

Archived production samples from lots that contained about 300 ppm Cl at the time of manufacture declined to as low as 58 ppm over almost 4 years of uncontrolled temperature, warehouse storage in HDPE 55 gallon barrels. However, ORP levels remained high throughout. Some remained unchanged over more than two years of storage; few declined more than 10%. Samples stored unsealed in small vessels (about 100 mL) showed precipitous declines in Cl ppm, losing approximately 90% of their Cl content in six months.

The findings demonstrate that long-lived stable unbuffered and uncontaminated HOCl is present in the representative HOCl solutions. Optimally stored and sealed these solutions may undergo minimal detectable changes upon prolonged storage even at high temperatures, with no degradation to chlorate or hypochlorite.

### Example 4

### Efficacy of Representative HOCl Solutions

The following describes the efficacy of representative HOCl solutions useful in the methods of the invention (i.e., stable, unbuffered HOCl solutions, BrioHOCL™) uncontaminated with other species of aqueous halogen in efficacy tests against a range of infectious agents, including fungal and bacterial spores and infectious proteins.

Table 1 shows the compilation of efficacy studies of representative HOCl solutions useful in the methods of the invention (BrioHOCL™) containing no other aqueous halogen species versus a variety of infectious agents. It is known that under some circumstances the molarity of the background inorganic salts can be an important determinant of the conformation of the proteinaceous targets of oxidation. Therefore, molar NaCl concentrations of some of these stable formulations may be contributing to the speed and potency of the disinfecting process for certain test agents.

TABLE 1
Compilation of results of efficacy determinations of representative HOCl
(BrioHOCL ™) solutions uncontaminated with any other aqueous halogen
species or extraneous additives versus a range of infectious agents.

| | Log | | | |
|---|---|---|---|---|
| | Reduction | | Testing | |
| Pathogen | Value | Elimination % | Date | Testing Site |
| Acinetobacter | 5.0 | >99.999% | 2 Jun. 2016 | Northwest Regional |
| baumannii | | | | Center of Excellence |
| | | | | for Biodefense & |
| | | | | Emerging Infectious |
| | | | | Diseases Research, |
| | | | | Univ of Washington |
| Aspergillus | 6.41 | >99.999% | 3 Aug. 2016 | Pacific Northwest |
| niger | | | | Microbiology |
| | | | | Services |
| Bacillus | 6.12 | >99.999% | 3 Aug. 2016 | Pacific Northwest |
| subtilis | | | | Microbiology |
| | | | | Services |
| Candida | 5.88 | >99.999% | 20 Nov. 2015 | Pacific Northwest |
| albicans | | | | Microbiology |
| | | | | Services |
| Coronavirus | 5.00 | >99.999% | 4 Mar. 2016 | School of Public |
| (Human, QC43) | | | | Health, Univ of |
| | | | | Washington (UW) |
| Enterobacter | >6.89 | >99.999% | 15 Jun. 2016 | Pacific Northwest |
| cloacae | | | | Microbiology |
| | | | | Services |
| Enterococcus | 6.07 | >99.999% | 20 Nov. 2015 | Pacific Northwest |
| faecalis (VRE) | | | | Microbiology |
| | | | | Services |
| Escherichia | 7.98 | >99.999% | 3 Aug. 2016 | Pacific Northwest |
| coli | | | | Microbiology |
| | | | | Services |
| Escherichia | 5.47 | >99.999% | 20 Nov. 2015 | Pacific Northwest |
| coli 0157 | | | | Microbiology |
| | | | | Services |
| Escherichia | >7.08 | >99.999% | 15 Jun. 2016 | Pacific Northwest |
| coli NDM-1 | | | | Microbiology |
| | | | | Services |
| Klebsiella | 7.63 | >99.999% | 20 Nov. 2015 | Pacific Northwest |
| pneumoniae | | | | Microbiology |
| | | | | Services |
| Listeria | Neg | >99% | 2 Mar. 2015 | Cascade Analytical |
| monocytogenes | culture | | | Inc. |
| Mold (fungus | Neg | >99% | 15 Apr. 2015 | Cascade Analytical |
| NOS) | culture | | | Inc, |
| MESA (Staph, | 5.0 | >99.999% | 2 Jun. 2016 | NW Regional COE |
| aureus) | | | | for Biodefense & |
| | | | | Emerging Infectious |
| | | | | Disease Research, UW |
| Polymicrobial | 3.41 | 99.96% | 15 Nov. 2016 | Pacific Northwest |
| biofilm | | | | Microbiology |
| | | | | Services |
| Prions (vCJD, | >6 | >99.999% | 29 Sep. 2016 | Rocky Mountain |
| others) | | | | Laboratories, US |
| | | | | National Institutes |
| | | | | of Health |
| Proteus vulgaris | >7.16 | >99.999% | 15 Jun. 2016 | Pacific Northwest |
| | | | | Microbiology |
| | | | | Services |
| Pseudomonas | 5.47 | >99.999% | 20 Nov. 2015 | Pacific Northwest |
| aeruginosa | | | | Microbiology |
| | | | | Services |
| Salmonella | 7.97 | >99.999% | 20 Nov. 2015 | Pacific Northwest |
| choleraesuis | | | | Microbiology |
| | | | | Services |
| Shigella | >6.75 | >99.999% | 15 Jun. 2016 | Pacific Northwest |
| flexneri | | | | Microbiology |
| | | | | Services |
| Staph | Roughly 2 | 99% | 11 May 2016 | Scientific Clinical |
| epidemidis | | | | Labs, Dubai |
| Yersinia | >6.29 | >99.999% | 15 Jun. 2016 | Pacific Northwest |
| enterocolitica | | | | Microbiology |
| | | | | Services |

Suspension test protocols for determination of efficacy in Table 1 used a modified ASTM E2315 Time/Kill test. Suspensions of cultured organisms of known concentrations were directly mixed with a volume of the HOCl test agent for a defined contact time. At the end of that time the activity of the test solution was terminated by addition of an excess of neutralizer. Plate counts of colony forming units were made after incubation at either room temperature or at 37° C., depending on the organism, to determine the extent of the inactivation of the target microbe using serial dilutions.

Full details of the measurement of the efficacy of BrioHOL™ versus infectious proteins are provided in Hughson, A. G., Race. B., Kraus, A., Sangaré, L. R., Robins, L., Contreras, L., Groverman, B. R., Terry, D., Williams. J., and Caughey, B. (2016), Inactivation of Prions and Amyloid Seeds with Hypochlorous Acid. PLoS Pathogens, 12(9), e1005914. http://doi.org/10.1371/journal.ppat.1005914, expressly incorporated herein by reference in its entirety. Briefly, Real Time Quaking Induced Conversion (RT-QuIC) assays were used to demonstrate that immersion in BrioHOCL™ eliminated all detectable prion-seeding activity for human Creutzfeldt-Jakob Disease (CJD) prions, bovine spongiform encephalopathy (BSE) prions, cervine chronic wasting disease (CWD) prions, and sheep scrapie and hamster scrapie prions, causing reductions of >10³ to 10⁶ fold in 5 minutes to 60 minutes of exposure. Transgenic mouse bioassays showed that all detectable hamster-adapted sheep scrapie infectivity in brain homogenates or on steel wires was eliminated. These results represent reductions of infectivity of approximately 10⁶ fold and 10⁴ fold, respectively. Inactivation of RT-QuIC activity correlated with free chlorine concentration in the HOCl solutions, and higher order aggregation and/or destruction of proteins generally, including prion proteins. Those preparations of unbuffered Briotech HOCl that contained approximately 2% NaCl showed superior efficacy over solutions that were isotonic with mammalian cells (i.e., approximately 0.85% NaCl), These solutions of unbuffered HOCl uncontaminated by the presence of other aqueous halogen species had similar effects on self-replicating amyloid proteins composed of human alpha synuclein and a fragment of human tau protein.

The attributes of the unbuffered HOCl solutions demonstrated in these studies are clearly novel and superior to commonly identified disinfecting capabilities of conventional aqueous halogen preparations, and additionally are superior to the sterilizing efficacy associated with certain chemical formulations relied upon in the stream of commerce today. The results overall not only meet the generally accepted criteria used by US and international regulatory agencies for characterization of the formulations as a sterilant, eliminating all forms of microbial life, but in addition are demonstrably capable of inactivating the most resistant of all infectious agents, the prion proteins associated with human and animal neurodegenerative diseases.

### Example 5

### Antimicrobial Properties of Representative HOCl Solutions

The following describes the antimicrobial properties of representative HOCl solutions useful in the methods of the invention (i.e., stable, unbuffered HOCl solutions, BrioHOCL™) versus resistant agents after prolonged storage of the solutions.

Test samples of BrioHOCL™ varying in age from the time of production from 3 to 34 months showed high degrees of efficacy in inactivating a range of target microbes, including spores of Bacillus subtilis (Table 2). Exposures as brief as 15-20 seconds were generally sufficient to produce LRVs in the 4-7 range, across the board, with the potency declining noticeably, but not seriously in the oldest materials tested. Aspergillus spores proved the least susceptible, though exposures of 60 seconds resulted in an LRV of >6 with the freshest, 3 month old sample of BrioHOCL™. Over time in storage the pH of the formulation trended upwards from the starting production-targeted level of 3.9 to about 5 by the second year in 55 gallon HDPE barrels (average of 6 samples).

TABLE 2
Tabulated results of the efficacy of representative HOCl
(BrioHOCL¹) solutions that had been aged for extended periods
before testing against highly resistant microbial organisms.

| | BrioHOCl | Contact | | |
|---|---|---|---|---|
| | Sample Age | Time | | |
| Microorganism | (months) | (seconds) | Cl ppm | LRV |
| Salmonella | 3 | 20 | 94 | 6.9 |
| choleraesuls | | | | |
| (ATCC [symbol] ) | | | | |
| Salmonella | 34 | 20 | 80 | 7.8 |
| choleraesuls | | | | |
| (ATCC [symbol] ) | | | | |
| Bacillus subtilis | 3 | 15 | 94 | 6.0 |
| spores ( [symbol] [symbol] ) | | | | |
| Bacillus subtilis | 10 | 15 | 240 | 6.1 |
| spores* ( [symbol] [symbol] ) | | | | |
| Bacillus subtilis | 34 | 15 | 80 | 3.9 |
| spores ( [symbol] [symbol] ) | | | | |
| Pseudomonas | 3 | 15 | 94 | 5.5 |
| aeruginosa | | | | |
| (ATCC [symbol] ) | | | | |
| Pseudomonas | 34 | 20 | 80 | 7.6 |
| aeruginosa | | | | |
| (ATCC [symbol] ) | | | | |
| Aspergillus niger | 3 | 60 | 94 | 6.5 |
| spores (ATCC [symbol] ) | | | | |
| Aspergillus niger | 34 | 60 | 80 | 4.0 |
| spores (ATCC [symbol] ) | | | | |
| Candida albicans | 3 | 15 | 94 | 6.8 |
| (ATCC [symbol] ) | | | | |
| Candida albicans | 34 | 30 | 58 | 3.9 |
| (ATCC [symbol] ) | | | | |

[symbol]

indicates data missing or illegible when filed

The results showed that aged BrioHOCL™ solutions, in the absence of other contaminating aqueous halogen species or any other extraneous additives, remained potently active as inactivators of disinfection-resistant spores of bacteria and fungi. High levels of inactivation were achieved in contact times of a few tens of seconds, even after storage periods of almost three years. These levels of microbial inactivation meet the criteria for characterization of the solutions as sterilants, resulting in the failure to survive of the most, resistant microbial life forms, the spores of anaerobic bacteria

### Example 6

### Efficacy of Representative HOCl Solutions Against Biofilm Microbial Populations

The following describes the efficacy of representative HOCl solutions useful in the methods of the invention (i e., stable, unbuffered HOCl solutions, BrioHOCL™) versus established biofilm microbial populations.

These experiments were conducted to measure the removal of established microbial biofilm populations in narrow bore polyurethane tubing following exposure to either static infusion of BrioHOCL™ or under conditions of flow. The solutions were prepared electrolytically and contained no extraneous additives or detectable aqueous halogen species other than HOCl. These adherent populations are known to be highly resistant to conventional antimicrobial disinfectants and antibiotic preparations. In the first experiment the exposure was static e., BrioHOCL™ solutions were infused into the lumen of tubing which had been allowed to develop extensive adherent biofilm populations) for a range of contact times. In the second the solution was allowed to flow over the adherent biofilm at approximately 1 mL/sec. After these exposures, residual populations were quantified as colony forming units per unit of surface area of the polyurethane tubing internal wall. Heterotrophic bacteria were preferentially cultured on. R2A medium at room temperature.

TABLE 3
Effect of static exposure of microbial biofilm populations established
on the luminal wall of polyurethane tubing to representative
HOCl solutions useful in the methods of the invention (i.e.,
stable, unbuffered HOCl solutions, BrioHOCL ™).

| | | | | % Reduc- | LRV |
|---|---|---|---|---|---|
| Sample | CFU/mL | Total CFU | CFU/cm² | tion/cm² | Reduction |
| Water in | 690,000 | NA | NA | NA | NA |
| Lumen | | | | | |
| Control | 360,000 | 3,600,000 | 764,331 | NA | NA |
| 5 Minute | 450 | 4,500 | 956 | 99.87 | 2.90 |
| Treatment | | | | | |
| 10 Minute | 140 | 1,400 | 298 | 99.96 | 3.41 |
| Treatment | | | | | |
| 20 Minute | 1,100 | 11,000 | 2,336 | 99.69 | 2.51 |
| Treatment | | | | | |
| 60 Minute | 240 | 2,400 | 510 | 99.93 | 3.17\| |
| Treatment | | | | | |

The results shown in Table 3 demonstrate that almost complete biofilm removal was achieved in 5 minutes.

TABLE 4
Effect of flowing representative HOCl solutions useful
in the methods of the invention (i.e., stable, unbuffered
HOCl solutions, BrioHOCL ™) at room temperature
through polyurethane tubing at the rate of about 1 mL/sec
on the populations of adherent microbial biofilm populations
at the end of the treatment times shown.

| | | | | % Reduc- | LRV |
|---|---|---|---|---|---|
| Sample | CFU/mL | Total CFU | CFU/cm² | tion/cm² | Reduction |
| Control | 141,000 | 1,410,000 | 374,014 | NA | NA |
| 1 Minute | 39 | 390 | 103 | 99.97 | 3.56 |
| Treatment | | | | | |
| 2 Minute | 52 | 520 | 138 | 99.96 | 3.44 |
| Treatment | | | | | |
| 3 Minute | 100 | 1,000 | 265 | >99.92 | 3.15 |
| Treatment | | | | | |
| 4 Minute | 25 | 250 | 66 | 99.98 | 3.76 |
| Treatment | | | | | |

The results shown in Table 4 demonstrate that almost complete biofilm removal was achieved after 1 minute of flow.

The results shown above illustrate the rapid, highly effective dislodging of resistant adherent heterotrophic bacterial populations, and marked disinfecting effect on the liberated microbial population.

### Example 7

### Preparation of Representative HOBr Solutions and Efficacy in Inactivation of Resistant Microbes

The following describes the preparation of a representative HOBr solution useful in the methods of the invention (i.e., stable, unbuffered HOBr solution) and its efficacy in inactivation of resistant microbes.

The conversion of a representative HOCl solution to a representative HOBr solution was accomplished rapidly such that in a few tens of seconds HOCl was no longer detectable spectroscopically in the starting HOCl solution (BrioHOCL™), and a new peak of HOBr is established. By adjusting the pH upwards with alkali the HOBr is instantly converted to OBr⁻ ions, which exhibit a characteristic peak in the UV range at 330 nm in aqueous solution.

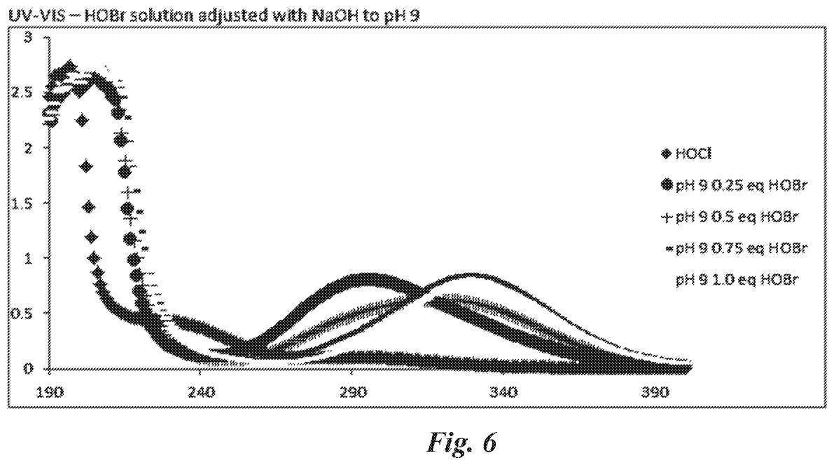

FIG. 6 is the UV/Vis absorption spectrum of a representative HOBr solution prepared as described above adjusted to pH 9 with sodium hydroxide.

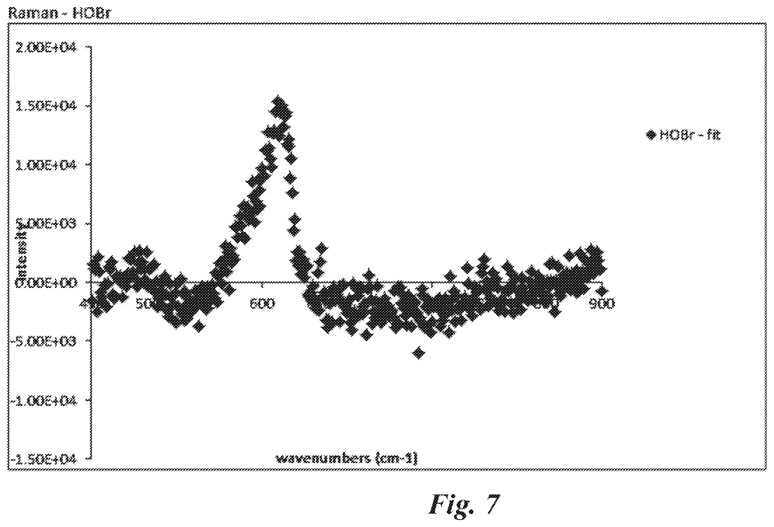

FIG. 7 is the Raman spectrum of a representative HOBr solution prepared as described above illustrating the characteristic waveform peak at 615 cm⁻¹. In the Raman spectra of these preparations there was no peak corresponding to HOCl and a new peak appeared at wavenumber 615 cm⁻¹ attributable to HOBr. This peak declined on storage at room temperature with a half-life of approximately 18 days.

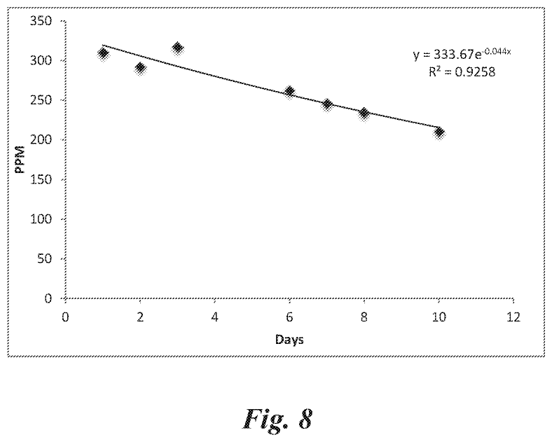

FIG. 8 illustrates titrable bromine (Br) (ppm) versus time of representative HOBr solutions prepared as described above after storage at room temperature in glass containers.

The relatively short storage life of HOBr contrasts sharply with the prolonged stability of HOCl under comparable circumstances. Nevertheless, the unbuffered HOBr solutions prepared in this way showed much greater stability than has been shown in the literature for conventionally prepared HOBr, typically made using bromide salt addition to aqueous chlorine solutions that contain various species of active Cl. Those kinds of HOBr preparations show decay of the active HOBr measured in minutes to hours, as compared to the several weeks of useful life shown in the experiments described herein. Test samples of HOBr containing no detectable HOCl by UV spectroscopy showed high degrees of efficacy in inactivating spores of Bacillus subtilis. Exposures as brief as 20 seconds to HOBr at approximately 25 ppm were sufficient to produce LRV of 6. In the same experimental protocol HOCl at 230 ppm was required to produce 6 LRV in the same contact time. As soon as the HOCl concentration used was below 230 ppm, the LRVs fell into the 2-4 range. At 25 ppm of HOCl there was no detectable effect on Bacillus spores in 20 seconds of contact. Inactivation of infectious proteins by HOBr reached comparable levels to those achieved using HOCl in tests using RTQuIC protocols.

The findings indicate the HOBr solutions so formed can provide potent antimicrobial activity against resistant organisms that may be practically useful in situations where the environmental pH is inimical to the presence of HOCl (e.g., at pH 8), but where the full potency of HOBr can be expected to be available due to its higher pKa. The activity in these test systems permit characterization of the HOBr solutions as sterilants, capable of inactivating all forms of microbial life, and in addition providing for the inactivation of infectious prion proteins.

### Example 8

### Antimicrobial Properties of Mists of Representative HOCl Solutions

The following describes the antimicrobial properties of mists of representative HOCl solutions useful in the methods of the invention (i.e., stable, unbuffered HOCl solutions, BrioHOCL™).

20 L of BrioHOCL™ was dispensed via a MF-1-001A Mist Fan Industrial Centrifugal Fogger 80,000 cu ft of air space in a closed facility known to harbor significant microbial contamination deposits of Pseudomonas and other environmental contaminants associated with use of the facility for food processing. Mist dispersal was at the rate of about 350,000 cu ft/hr. Operators remained within the misted air space during the dispersal, and experienced no adverse effects. Active Cl was detected throughout the facility by placement of Cl-sensitive test strips at each corner of the enclosed space prior to misting, and all showed conversion at the level of 200 ppm Cl at the end of the misting process. Follow-up swab cultures for bacteria on walls and ducts surfaces demonstrated that the mist dispersion method for the HOCl solution effectively distributed sufficient HOCl to bring about high level inactivation rates for microbial contaminants. The results furthermore indicate that misted HOCl solutions (BrioHOCL™) not only disperse sufficient active Cl to effect disinfecting decontamination, but do so in a manner that is compatible with operator safety, even when personnel remain fully exposed to the active mist over the course of the procedure.

### Example 9

### Safety of Representative HOCl and HOBr Solutions

The following describes the safety of representative HOCl and HOBr solutions useful in the methods of the invention (i.e., unbuffered HOCl solutions, BrioHOCL™; unbuffered HOBr solutions, BrioHOBR™).

BrioHOCL™ and BrioHOBR™ were applied to human skin and mucous membranes.

BrioHOCL™ from lots comparable to those used in antimicrobial studies described herein was provided to 50 people for spray application to healthy skin or mucous membranes, or to a variety of skin and/or mucous membrane lesions, over a period of 12 months. Use-patterns were selected entirely at the discretion of the recipients. There were no reports of adverse reactions of any kind from any applications, some of which involved multiple uses per day, over periods of days to weeks. A number of clinical conditions, including those resulting from infectious processes, were reported to be ameliorated or eliminated by dermal exposure to BrioHOCL™. The results indicate that repeated exposure of human dermal and mucosal epithelia is entirely safe, and may contribute beneficially to the resolution of certain clinical conditions.

Freshly prepared HOBr solutions was made by addition of an equivalent of NaBr to a solution of HOCl containing 200 ppm of Cl. This solution was also applied to human skin and mucous membranes without any indication of adverse effects on these epithelial surfaces.

While illustrative embodiments have been illustrated and described, it will be appreciated that various changes can be made therein without departing from the spirit and scope of the invention.

1-10. (canceled)

11. A method for inactivating an infectious protein or a microbial pathogen, comprising contacting an infectious protein or a microbial pathogen with a bufferless, electrolyzed, aqueous hypohalous acid composition.

12. The method of claim 11, wherein the infectious protein is an infectious self-replicating protein.

13. The method of claim 11, wherein the infectious protein is a prion.

14. The method of claim 13, wherein the prion is an agent of Creutzfeldt Jakob Disease, Bovine Spongiform Encephalopathy, Chronic Wasting Disease, Scrapie, Alzheimer's Disease, Parkinson's Disease, and Amyotrophic Lateral Sclerosis.

15. (canceled)

16. The method of claim 11, wherein the microbial pathogen is a Gram negative bacterium.

17. The method of claim 11, wherein the microbial pathogen is a Gram positive bacterium.

18. The method of claim 11, wherein the microbial pathogen is a fungus or a virus.

19. (canceled)

20. The method of claim 1, wherein the composition is a solution, a spray or fog or mist or aerosol of droplets, a gel, or a viscous liquid.

21-23. (canceled)

24. The method of claim 1, wherein the hypohalous acid composition is a hypochlorous acid composition or a hypobromous acid composition.

25. The method of claim 1, wherein the hypohalous acid composition is an aqueous hypochlorous acid composition having a hypochlorous acid concentration from about 5 to about 500 mg/L, a pH from about 3.2 to about 6.0, an oxidative reduction potential (ORP) of about +1000 millivolts, and containing from about 0.85% to about 2.0% by weight chloride salt based on the total weight of the composition.

26-28. (canceled)

29. The method of claim 1, wherein the hypohalous acid composition is an aqueous hypobromous acid composition having a hypobromous acid concentration from about 10 to about 300 mg/L, a pH from about 3 to about 8.5, an oxidative reduction potential (ORP) of about +1000 millivolts, and containing from about 0.85% to about 2.0% by weight chloride salt based on the total weight of the composition.

30. (canceled)

31. The method of claim 25, wherein the chloride salt is an aqueous soluble chloride salt selected from sodium chloride, potassium chloride, magnesium chloride, and ammonium chloride.

32. (canceled)

33. The method of claim 25, wherein the composition contains about 2.0% by weight chloride salt based on the total weight of the composition.

34-39. (canceled)

40. A bufferless, electrolyzed, aqueous hypohalous acid composition, comprising a hypohalous acid and a chloride salt in an amount from about 0 to about 2.0% by weight based on the total weight of the composition.

41. The composition of claim 40, wherein the chloride salt is an amount from about 0.85 to about 2.0% by weight based on the total weight of the composition.

42. The composition of claim 40, wherein the hypohalous acid is hypochlorous acid or hypobromous acid.

43. The composition of claim 40, wherein the composition comprises hypochlorous acid at a concentration from about 5 to about 500 mg/L, and has a pH from about 3.2 to about 6.0, and an oxidative reduction potential (ORP) of about +1000 millivolts.

44-46. (canceled)

47. The composition of claim 40, wherein the composition comprises hypobromous acid at a concentration from about 10 to about 300 mg/L, and has a pH from about 3 to about 8, and an oxidative reduction potential (ORP) of about +1000 millivolts.

48. (canceled)

49. The composition of claim 40, wherein the chloride salt is an aqueous soluble chloride salt selected from sodium chloride, potassium chloride, magnesium chloride, and ammonium chloride.

50-55. (canceled)

56. The composition of claim 40 formulated as a solution, a spray or fog or mist or aerosol of droplets, a gel, or a viscous liquid.
